(19) United States
(12) Patent Application Publication
(10) Pub. No.: US 2006/0047257 A1
(43) Pub. Date: Mar. 2, 2006

(54) Extensible absorbent core and absorbent article

(76) Inventors: Maria Raidel, Fuerth (DE); Franz Aschenbrenner, Kastl (DE); Jan Ullmann, Nuernberg (DE)

Correspondence Address: KIMBERLY-CLARK WORLDWIDE, INC., 401 NORTH LAKE STREET, NEENAH, WI, 54956, US

(21) Appl. No.: 10/930,226

(22) Filed: Aug. 31, 2004

(51) Int. Cl. A61F 13/15

(52) U.S. Cl. 604/383

(57) ABSTRACT

The present invention provides an absorbent article, such as a sanitary napkin or catamenial napkin, wherein the absorbent article has a longitudinal direction and a lateral direction, the absorbent article has: a) a liquid-permeable top layer, wherein the top layer has an extensibility in lateral direction of at least 30% to 70% at 4N/50 mm, b) a liquid-impermeable backing layer laminated to the top layer, wherein the backing layer has a higher extensibility in lateral direction than in longitudinal direction and the extensibility in lateral direction is at least between 5% and 40% at 4N/50 mm, and c) an absorbent core positioned between the top layer and the backing layer, wherein the absorbent core has a plurality of non-linear slits and/or linear slits at an acute angle relative to the longitudinal direction through the absorbent core. The absorbent core may be a variety of materials, however one particular example is an absorbent core with a layer of material having a basis weight of between 20 g/m² to 1000 g/m² and is an airlaid fiber layers, coform fiber layers, meltblown fiber layers or laminates thereof and laminates of airlaid fiber layers, coform fiber layers or meltblown fiber layers or laminates thereof with a nonwoven support. The absorbent core generally will have a ratio of tensile strength in longitudinal direction and resistance to tear in lateral direction has a value in the range from 3.5:1 to 6:1. The invention further provides a punching die for forming slits in an nonwoven absorbent core material and a process for forming an absorbent article using said punching die.

### FIELD OF THE INVENTION

The present invention relates to disposable absorbent articles such as sanitary napkins, panty liners or incontinence pads and absorbent cores used therein. The present invention further relates to a process of manufacturing an absorbent core or absorbent article, as well as a punching die used in the process.

### BACKGROUND OF THE INVENTION

Disposable absorbent articles, such as sanitary napkins worn by women, must fulfill numerous requirements in order to be considered satisfactory for use. From the standpoint of the consumer using the disposable absorbent article, efficient absorption and retention of liquid and other discharges from the human body must be provided for, and possible soiling of the wearer's body or the wearer's garment should be avoided. Additionally, the product should be comfortable to wear. The absorbent article should therefore be able to individually conform to the shape of the wearer's body, so that the wearer is not hindered by the absorbent article. Ideally, the absorbent article should not be perceived by the wearer at all when worn. On the other hand, the absorbent article should not be too soft and deformable, otherwise bunching, twisting or roping of the absorbent article or lumping of absorbent core may occur, when the article is worn. This would seriously deteriorate the performance of the absorbent article and influence negatively the wearing comfort. Furthermore, the absorbent article and especially the absorbent core must have sufficient structural integrity to withstand the forces exerted onto the absorbent article when worn. At the same time the absorbent article should be thin and therefore inconspicuous when worn. From the manufacturer's point of view, the materials used to produce the absorbent article must be suitable for mass production at very high production rates. The materials used to produce the article must therefore be easily obtainable and of relatively low cost. Furthermore, the materials must be easy to process at high speeds, at which considerable forces and tensions, especially in the machine direction, are exerted onto these materials.

In order to increase the wearing comfort of sanitary napkins it has been suggested in the art to provide these articles with stretchability or increased flexibility. Such sanitary napkins are, for instance described, in U.S. Pat. No. 5,674,212 issued to Osborn, III. There it is described that the longitudinal extensibility of an absorbent core can be increased by slitting or partially slitting the absorbent core or providing it with diamond shaped apertures. In European patent application EP 0293 208 to Uchida et al. it is described that the flexibility of a compressed cellulose-based sponge sheet can be increased by providing the absorbent core with a pattern of slits. U.S. Pat. No. 5,397,316 describes slitted absorbent members for aqueous body fluids, wherein the absorbent members are formed of polymeric foam materials, which are expandable.

Excessive extensibility of an absorbent core in longitudinal direction, which as a rule corresponds to the machine direction, will make it very difficult to use such an absorbent core in a production line designed for very high production rates (high speeds). Furthermore, for the absorbent article to be able to conform to the body of the wearer, the absorbent core should be sufficiently extensible in both the longitudinal and lateral direction, while maintaining sufficient strength in both directions. At the same time care must be taken not to reduce the flexural resistance of the absorbent article too far, because otherwise the article will bunch, twist or rope or lumps will form in the absorbent core when worn. Additionally the articles might no longer be accepted by the consumer if it feels too flexible, i.e. flimsy or flabby, thereby creating the impression that the article will not provide sufficient protection against soiling of the garment to be protected.

Furthermore, care should be taken that the formation of slits in the absorbent core does not impair the efficiency of liquid intake, liquid distribution and liquid retention, due to the quick passage of liquid through the absorbent core via the cuts or apertures formed therein.

It would therefore be desirable to provide an absorbent article which is extensible and highly conformable to the body of the wearer, while at the same time maintaining sufficient structural strength to avoid bunching, twisting or roping and formation of lumps, making efficient usage of the absorbent capacity of the absorbent core, and which can be produced at high production rates (high speeds) at relatively reasonable or low cost, preferably from commercially available materials.

### SUMMARY OF THE INVENTION

The present invention provides an absorbent article which is extensible and highly conformable to the body of the wearer. Furthermore the present invention provides an extensible and highly conformable absorbent core which efficiently utilizes the absorbent capacity of the absorbent core.

The present invention solves the above outlined problems by providing an absorbent article, an absorbent core for use in an absorbent article, a process for manufacturing the absorbent core and the absorbent article and a punching die used in the process for manufacturing the absorbent core and the absorbent article as defined in the independent claims. Further embodiments and features of the different aspects of the present invention become apparent from the dependent claims, the description and the figures appended thereto.

In a first aspect, the present invention provides an absorbent article, such as a sanitary napkin or catamenial napkin, wherein the absorbent article has a longitudinal direction and a lateral direction. The absorbent article has: a) a liquid-permeable top layer, wherein the top layer is extensibility in lateral direction, b) a liquid-impermeable backing layer laminated to the top layer, wherein the backing layer has a higher extensibility in lateral direction than in longitudinal direction and c) an absorbent core positioned between the top layer and the backing layer. The extensibility of the top layer in the lateral direction is at least 30% to 70% at 4N/50 mm and the backing layer has an extensibility in lateral direction is at least 5% to 40% at 4N/50 mm. The absorbent core is an absorbent nonwoven web which has a plurality of non-linear slits, linear slits at an acute angle relative to the longitudinal direction and mixtures thereof through the absorbent core. The nonwoven web of the absorbent core may have a basis weight of between 20 g/m² to 1000 g/m² and may be selected from the group consisting of airlaid fiber layers, coform fiber layers, meltblown fiber layers or laminates thereof and laminates of airlaid fiber layers, coform fiber layers or meltblown fiber layers or laminates thereof with a nonwoven support. The ratio of the tensile strength of the absorbent core in longitudinal direction and the tensile strength of the absorbent core in lateral direction desirably has a value in the range from 3.5:1 to 6:1.

The combined use of extensible materials for the top layer, backing layer and absorbent core provides for a highly extensible absorbent article. The extensibility of the absorbent core material is further increased by the provision of the non-linear slits therein. The ratio of resistance to tearing in longitudinal direction (corresponding to the machine direction) and in lateral direction (corresponding to the cross machine direction) of the slitted absorbent core makes it possible for the slitted absorbent core to be processed at high speeds in a production line. The slitted absorbent core material used in the present invention still maintains a sufficient flexural resistance and integrity to avoid bunching, twisting or roping of the absorbent article or formation of lumps or clusters in the absorbent core when worn, or to be considered to be too flexible (flabby, flimsy) by the consumers.

In a second aspect, the present invention provides an absorbent core for use in an absorbent article having a longitudinal direction and a lateral direction, wherein the absorbent core is made of a nonwoven absorbent material and has a pattern of slits formed therethrough, wherein each slit is formed by removing a section of the nonwoven material from the absorbent core and simultaneously increasing the density of the nonwoven material in the areas of the absorbent core adjacent to the removed section of the slit. According to the present invention, the nonwoven absorbent material can be made from airlaid, coform fibers and/or meltblown fibers.

By providing these specific slits in the absorbent core, the extensibility and flexibility of the absorbent core is increased compared to original non-slitted material. This is achieved not only by dividing the absorbent material in the area of the slits, but additionally by removing absorbent material from within the area of the slit, for instance as a thin strip of material corresponding to the respective form of the strip. Additionally, the nonwoven material in the areas of the absorbent core adjacent to the removed sections of the slit are densified, thereby defining areas of increased capillarity in the nonwoven material. This enhances the liquid transportation along the slits formed in the nonwoven material and leads to an improved liquid intake, distribution, and retention in the absorbent core. In case the absorbent article is a feminine hygiene product like a sanitary napkin, a pad, or a panty liner, the absorbent core has improved menses handling properties.

To form these special slits in the absorbent core to be used in the absorbent article, the present invention provides as third and fourth aspects of the present invention a special punching die for forming slits in an absorbent core for use in an absorbent article, as well as a process for manufacturing an absorbent core for use in an absorbent article. The punching die or punching tool according to the present invention has at least one plurality of punching elements for forming a pattern of slits in said absorbent core, each punching element comprising a blade section that has a special blade geometry which punches a thin slit out of the absorbent core material and at the same time densifies the areas of the absorbent core adjacent to the punched out slit areas, i.e. the small holes created by the punching tool or die.

In the process according to the present invention, a web of a nonwoven material for forming the absorbent core is provided and a pattern of slits is formed in the web using the above described punching die. The present invention further provides absorbent cores obtainable by this method as well as absorbent articles using such absorbent cores.

### BRIEF DESCRIPTION OF THE DRAWINGS

FIG. 1. is an exploded view of a first embodiment of an absorbent article according to the present invention.

FIG. 2 is an exploded view of a second embodiment of an absorbent article according to the present invention.

FIG. 3 is a schematic representation of a section of a pattern of slits in an absorbent core.

FIG. 4 a-d show further possible patterns of slits for use in the present invention.

FIG. 5 shows a first embodiment of the punching die according to the present invention.

FIG. 6 shows a cross section of the area denoted “Y” in FIG. 5.

FIG. 7 shows a projection of the outer surface of the punching die according to FIG. 5 onto a plane.

FIG. 8 shows a schematic representation of selected steps of the process for forming an absorbent article according to the present invention

### DEFINITIONS

As used herein with reference to the present invention, the term “disposable” includes being disposed after use and not intended to be washed and reused.

As used herein with reference to the present invention, the term “layer” when used in the singular can have the dual meaning of a single element or a plurality of elements.

As used herein with reference to the present invention, the term “liquid” means a non-particulate substance and/or material that flows and can assume the interior shape of a container into which it is poured or placed.

As used herein with reference to the present invention, the term “longitudinal” means having the longitudinal axis in the plane of the article and is generally parallel to a vertical plane that bisects a standing wearer into left and right body halves when the article is worn. The “lateral” axis lies in the plane of the article generally perpendicular to the longitudinal axis, i.e., so that a vertical plane bisects a standing wearer into front and back body halves when the article is worn.

As used herein, the term “machine direction” or “MD” means the length of a fabric in the direction in which it is produced. This direction as a rule essentially corresponds to the above defined longitudinal direction.

The term “cross machine direction”, “cross direction” or “CD” means the width of fabric, i.e. a direction generally perpendicular to the MD. In line with the above definitions, the cross machine direction as a rule essentially corresponds with the lateral direction of the absorbent core.

As used herein with reference to the present invention, the term “conjugate fibers” refers to fibers that have been formed from at least two polymers extruded from separate extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement wherein one polymer is surrounded by another or may be a side by side arrangement, a pie arrangement or an “islands-in-the-sea” arrangement. Conjugate fibers are taught in U.S. Pat. No. 5,108,820 to Kaneko et al., U.S. Pat. No. 5,336,552 to Strack et al., and U.S. Pat. No. 5,382,400 to Pike et al. For two component fibers, the polymers may be present in ratios of 75/25, 50/50, 25/75 or any other desired ratios. The fibers may also have shapes such as those described in U.S. Pat. No. 5,277,976 to Hogle et al., and U.S. Pat. Nos. 5,069,970 and 5,057,368 to Largman et al., hereby incorporated by reference in their entirety, which describe fibers with unconventional shapes.

As used herein with reference to the present invention, the term “biconstituent fibers” refers to fibers that have been formed from at least two polymers extruded from the same extruder as a blend. Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Biconstituent fibers are sometimes also referred to as multiconstituent fibers. Fibers of this general type are discussed in, for example, U.S. Pat. No. 5,108,827 to Gessner. Bicomponent and biconstituent fibers are also discussed in the textbook Polymer Blends and Composites by John A. Manson and Leslie H. Sperling, copyright 1976 by Plenum Press, a division of Plenum Publishing Corporation of New York, IBSN 0-306-30831-2, at pages 273 through 277.

As used herein with reference to the present invention, the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 35 microns. The fibers may also have shapes such as those described in U.S. Pat. No. 5,277,976 to Hogle et al., U.S. Pat. No. 5,466,410 to Hills and U.S. Pat. Nos. 5,069,970 and 5,057,368 to Largman et al., which describe fibers with unconventional shapes.

As used herein with reference to the present invention, the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers that may be continuous or discontinuous, are generally smaller than 10 microns in average diameter, and are generally tacky when deposited onto a collecting surface.

“Airlaying” is a well-known process by which a fibrous nonwoven layer can be formed. In the airlaying process, bundles of small fibers having typical lengths ranging from about 3 to about 52 millimeters are separated and entrained in an air supply and then deposited onto a forming screen, usually with the assistance of a vacuum supply. The randomly deposited fibers then are bonded to one another using, for example, hot air, a spray adhesive, or a binder. Examples of airlaying technology can be found in U.S. Pat. Nos. 4,494,278, 5,527,171, 3,375,448 and 4,640,810. The term “airlaid fiber layer” as used herein with reference to the present invention relates to layers obtained by means of an airlaying process.

As used herein with reference to the present invention, the term “coform” means a process in which at least one meltblown diehead is arranged near a chute through which other materials are added to the web while it is forming. Such other materials may be pulp, superabsorbent or other particles, natural polymers (for example, rayon or cotton fibers) and/or synthetic polymers (for example, polypropylene or polyester) fibers, for example, where the fibers may be of staple length. Coform processes are shown in commonly assigned U.S. Pat. No. 4,818,464 to Lau and U.S. Pat. No. 4,100,324 to Anderson et al. Webs produced by the coform process are generally referred to as coform materials.

“Carded web” refers to webs that are made from staple fibers that are sent through a combing or carding unit, which opens and aligns the staple fibers in the machine direction to form a generally machine direction-oriented fibrous nonwoven web. The web is then bonded by one or more of several known bonding methods.

Bonding of nonwoven webs may be achieved by a number of methods; powder bonding, wherein a powdered adhesive or a binder is distributed through the web and then activated, usually by heating the web and adhesive with hot air; pattern bonding, wherein heated calender rolls or ultrasonic bonding equipment are used to bond the fibers together, usually in a localized bond pattern, though the web can be bonded across its entire surface if so desired; through-air bonding, wherein air which is sufficiently hot to soften at least one component of the web is directed through the web; chemical bonding using, for example, latex adhesives that are deposited onto the web by, for example, spraying; and consolidation by mechanical methods such as needling and hydroentanglement.

“Personal care product” means diapers, training pants, absorbent underpants, adult incontinence products, swim wear, bandages and other wound dressings, and feminine hygiene products.

“Feminine hygiene products” means sanitary napkins, pads, and panty liners.

“Target area” refers to the area or position on a personal care product where an insult is normally delivered by a wearer.

### DETAILED DESCRIPTION OF THE INVENTION

In the following the present invention will be described especially with reference to sanitary napkins but is not limited to those. It is also applicable to other absorbent articles such as personal care products.

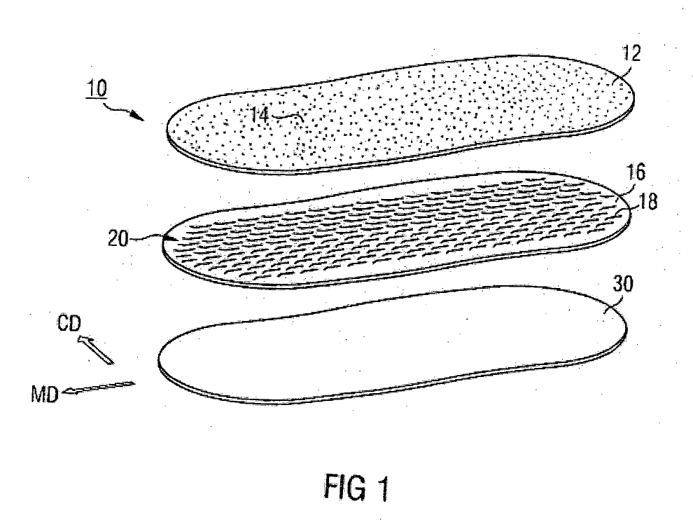

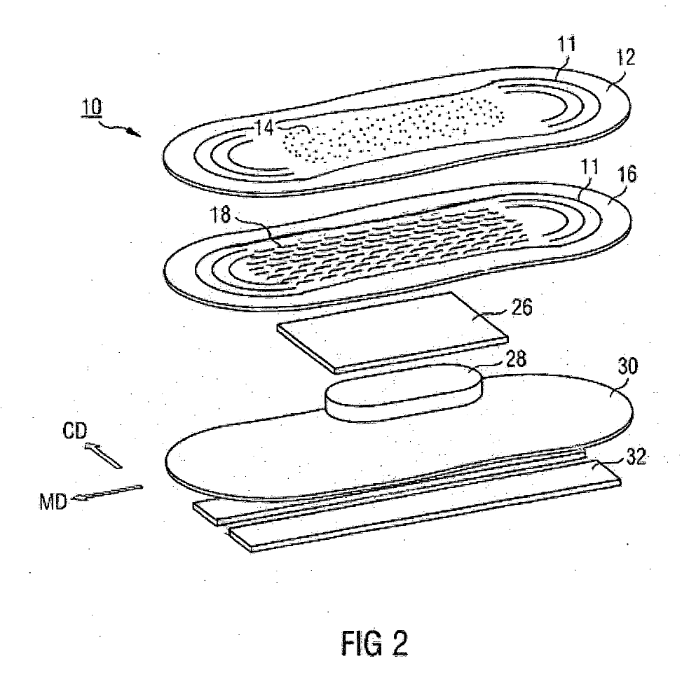

FIG. 1 and FIG. 2 show a first and a second embodiment of the absorbent article 10 according to the first aspect of the present invention. It is to be understood within the present application that all figures show selected embodiments comprising one or more aspects of the present invention and that the embodiments shown in these figures are non-limiting embodiments by means of which selected properties or selected components or features of the absorbent articles are demonstrated. The absorbent article according to the first aspect of the present invention has a longitudinal direction, corresponding to the machine direction MD, and a lateral direction, corresponding to the cross machine direction CD. The cross machine direction is perpendicular to the machine direction. The absorbent articles 10 comprise a liquid-permeable top layer 12, wherein the top layer 12 may have an extensibility in lateral direction of at least 30% to 70% at 4N/50 mm. The tensile strength of the top layer material can be determined following the procedures outlined in the tensile test on strips of textile fabrics of DIN 53 857 on a strip of material having a width of 50 mm and measured without a pre-load force. The top layer 12 is the layer facing the wearer's body when the absorbent article is in use. The top layer 12 can be made of a material that permits the passage of fluid without drawing the fluid horizontally in parallel to the top layer to any great extent. In addition, the top layer should retain little or no fluid in the structure, so that a relatively dry surface is provided next to the wearer's skin. In general, the top layer is a single layer of a material with a width sufficient to cover the surface of an absorbent core positioned underneath it. The top layer 12 preferably extends to the longitudinal edges of the absorbent article and is bonded to a liquid-impermeable backing layer 30. The top layer 12 may be bonded to the backing layer 30 using any known method which does not leave any hard or uncomfortable residues that would annoy the wearer. Those skilled in the art are familiar with methods of bonding the various materials and for bonding other possible materials in the absorbent article according to the present invention, including the use of pressure-sensitive adhesives, hot-melt adhesives, two-sided adhesive sheets, ultrasonic welding and heat sealing, to name but a few. Adhesives such as hot-melt adhesives may be used uniformly or in the form of a continuous or non-continuous layer.

The top layer 12 may be manufactured from materials known in the art. Known materials include, for example, card weaves and spunbonded nonwovens made of polyester, polypropylenes, polyethylenes, nylon or other heat-bonded fibers. Other polyolefins such as copolymers of polypropylene and polyethylene, linear, low-density polyethylene fiber nonwovens which are finely perforated or mesh-like materials are also suitable. Other suitable materials include composite materials of polymers and nonwoven material. The composite layers are usually formed by extrusion of the polymer on a layer of spunbonded nonwoven to form an integral layer. These materials may contain pigments, such as titanium dioxide to improve the masking properties of the top layer 12 or other additives to improve the properties of the material, like substances improving the hydrophilic properties of the top layer, e.g. surfactants.

In a non-limiting embodiment of the absorbent article according to the present invention, the top layer 12 is made of a carded nonwoven web made of polypropylene fibers, which generally has a basis weight of from about 18 to about 40 g/m². In a specific embodiment, the basis weight is 27 g/m². Alternatively, the top layer 12 may be made of a spunbonded nonwoven made of polypropylene fibers and generally having a basis weight of from about 18 to about 40 g/m². Top layer 12 may comprise a multitude of perforations 14, which may be oval shaped, square shaped or circular shaped, or of any other suitable shape, in order to facilitate the uptake of liquid through the top layer, especially highly viscous components of such a liquid. The top layer may be completely perforated or may only be perforated in selected areas thereof. In the specific embodiment shown in FIG. 1, the perforations are formed throughout the surface of the top layer 12. In the embodiment shown in FIG. 2, the perforations 14 are only situated in the central area of the top layer 12, corresponding to the target area of the absorbent article. Generally, the perforations 14 could also be present within a central strip extending along the complete length of the absorbent article or uniformly distributed over the whole area of the top layer 12. The perforations may be arranged randomly or regularly in a pattern.

Alternatively, the liquid-permeable top layer 12 may also consist of a perforated film made from a liquid-impermeable material, such as polyethylene or polypropylene.

The top layer 12 may further comprise embossing lines for adapting the shape of the absorbent article to the shape of the wearer's body and to improve its conformability to the body of the wearer. Such embossing lines can be formed using conventional techniques. If embossing lines are present in the top layer 12, the underlying absorbent core 16 will usually also comprise corresponding embossing lines because such embossing lines are usually formed in a single embossing step during the manufacturing of the absorbent article.

The absorbent articles 10 shown in FIGS. 1 and 2 further comprise a liquid-impermeable backing layer 30 laminated to the top layer 12, wherein the backing layer 30 has a higher extensibility in lateral direction than in longitudinal direction. The extensibility in lateral direction may have a value of between at least 5% and 40% at 4N/50 mm as determined following the procedures of the tensile test on strips of textile fabrics outlined in DIN 53 857 on a strip of material having a width of 50 mm and measuring without a pre-load force. The backing layer 30 may be made of any suitable material that is sufficiently liquid-impermeable and exhibits the required extensibility. The backing layer may for instance consist of a polymeric film having the desired extensibility. The backing layer 30 may allow atmospheric vapor and moisture to pass through the absorbent article while preventing body fluid from passing through. Suitable extensible materials are for instance mentioned in U.S. Pat. No. 4,166,464, the disclosure of which is fully incorporated herewith. Suitable polymer materials for use as the backing layer are, for instance, Kraton 1107, a thermoplastic elastomeric A-B-A (styrene-isoprene-styrene, S-I-S) copolymer or Solprene 420, which is also a S-I-S copolymer or mixtures thereof. Furthermore, a layer made of different types of polyethylene having different densities may be used. Additionally, microembossed polyethylene or polypropylene films of films of polyolefin foams may be used.

Both the top layer 12 and the backing layer 30 may comprise wing sections located at the longitudinal sides of the absorbent article. Such wing sections are commonly used in the art for attachment of sanitary napkins to the wearer's undergarment.

The absorbent articles according to the present invention further have an absorbent core 16 positioned between the top layer 12 and the backing layer 30. The absorbent core may be a layer of material having a basis weight of between 20 g/m² to 1000 g/m². Generally, the absorbent core is an absorbent nonwoven web. Suitable nonwoven webs are those nonwoven webs which are moisture stable and wettable. By “moisture stable”, it is meant a nonwoven web which does not loose its integrity when the nonwoven web becomes wetted, by a fluid. Exemplary absorbent nonwoven webs include, for example, nonwoven webs of airlaid fiber layers, coform fiber layers, meltblown fiber layers or laminates thereof and laminates of airlaid fiber layers, coform fiber layers or meltblown fiber layers or laminates thereof with a nonwoven support. Therein, the absorbent core comprises a plurality of non-linear slits therethrough, and the ratio of the tensile strength of the absorbent core in longitudinal direction and the tensile strength of the absorbent core in lateral direction has a value in the range from 3.5:1 to 6:1. In specific embodiments of the present invention this ratio is selected to be in the range between 4.5:1 to 5.6:1.

The slitted absorbent core used in the present invention should maintain a sufficient flexural resistance to avoid bunching of the absorbent core or formation of lumps therein when worn, or for it to be considered to be too flexible (flabby, flimsy) by the consumers. In specific embodiments of the present invention, the flexural resistance of the slitted absorbent core is selected to have a value in the range of about 0.5 N to 1 N as determined by the circular bending procedure described, for instance, in European Patent EP 0336578 corresponding to U.S. Pat. No. 4,950,264 to Osborn, III.

The absorbent core 16 is generally made of one or more materials which together are essentially absorbent, hydrophilic, compressible, adaptable and non-irritating for the skin of the wearer. Suitable materials are well known in the art and include, for example, various natural or synthetic fibers, cellulose fibers, regenerated cellulose or cotton fibers or a blend of cellulose and other fibers or polyethylene or polypropylene fibers, or biconstituent fibers. In a specific embodiment, polypropylene fibers may be used.

In a further embodiment of present invention, the absorbent core material has a basis weight up to about 800 g/m² and generally between 60 g/m² and 500 g/m², desirably between 100 g/m² and 200 g/m², or, more specifically, between 150 and 180 g/m².

The nonwoven support used in the absorbent core material may be a spunbond fiber layer made of polypropylene fibers and having a basis weight in the range of 10 to 40 g/m², desirably 10 to 25 g/m².

In a further embodiment of the present invention, the absorbent core material may be a laminate of an airlaid fiber layer or coform fiber layer with a basis weight of 140 g/m² to 170 g/m² and a spunbond fiber layer with a basis weight in the range of 10 to 40 g/m², specifically 10 to 25 g/m². When using a laminated absorbent core, the core is usually arranged in the absorbent core in such a manner that the support layer faces in the direction of the top layer, i.e. towards the wearer.

The absorbent core material may be an airlaid material, comprising a mixture of at least 70 wt.-% cellulose fibers, from about 2 to 6 wt-% polyethylene powder, 5 to 25 wt-% bicomponent or conjugate fibers, and optionally up to 2 wt-% of a latex binder, wherein the sum of the weight percentages of the different components present in the material adds up to 100 wt-%. Alternatively, the absorbent core material may be a coform material containing between 30 and 50 wt-% polypropylene and between 50 and 70 wt-% cellulose, wherein the sum of the weight percentages of the different components present in the material adds up to 100 wt-%. Specifically, the coform material may contain between 30 and 40 wt-% polypropylene and between 60 and 70 wt-% cellulose. In a further embodiment, the coform material contains 40 wt-% polypropylene and 60 wt-% cellulose. The absorbent material may further comprise additives to improve its properties, such as surfactants.

To improve the extensibility of the absorbent core while maintaining sufficient flexural resistance of the absorbent material, a pattern of non-linear slits and/or linear slits which are at an acute angle relative to the longitudinal direction is cut or punched into the absorbent core. The pattern of non-linear slits or linear slits which are at an acute angle relative to the longitudinal direction of the absorbent article should significantly decrease the tensile strength of the absorbent material in both the longitudinal direction (corresponding to the MD) and lateral direction (corresponding to the CD) while still maintaining sufficient stability and durability for the absorbent material to be processed at high speeds in a production line. At the same time, as already outlined above, the flexural resistance should not be decreased to much, otherwise the possibility of formation of lumps or bunching exists, or the absorbent article might be perceived by the consumer as being too flexible (flimsy, flabby). A reduction of the flexural resistance by between 40 and 70%, and more specifically by between 60 and 70%, compared to the non-slit material is considered to be specially suitable.

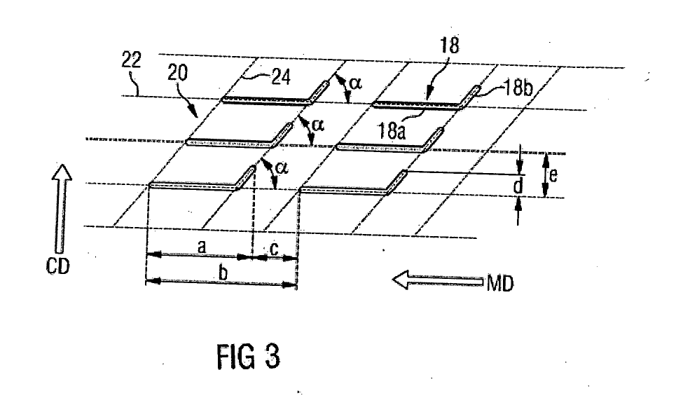

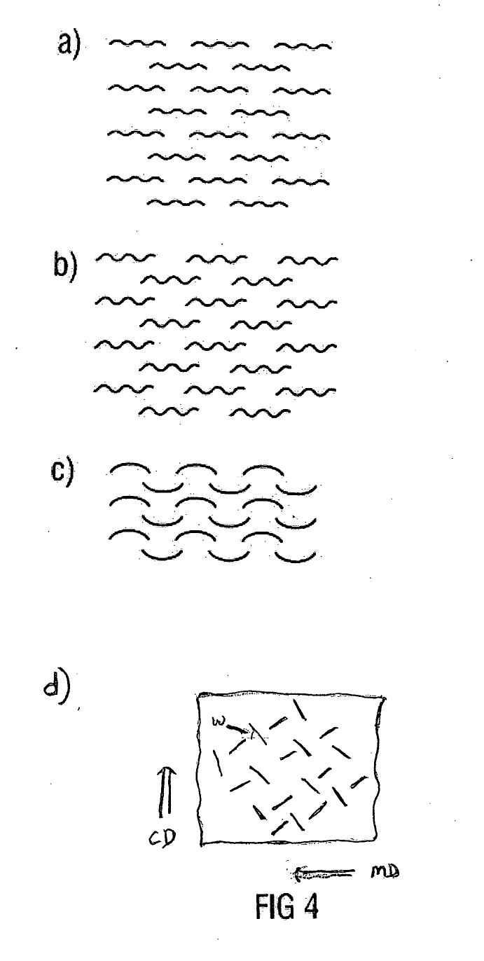

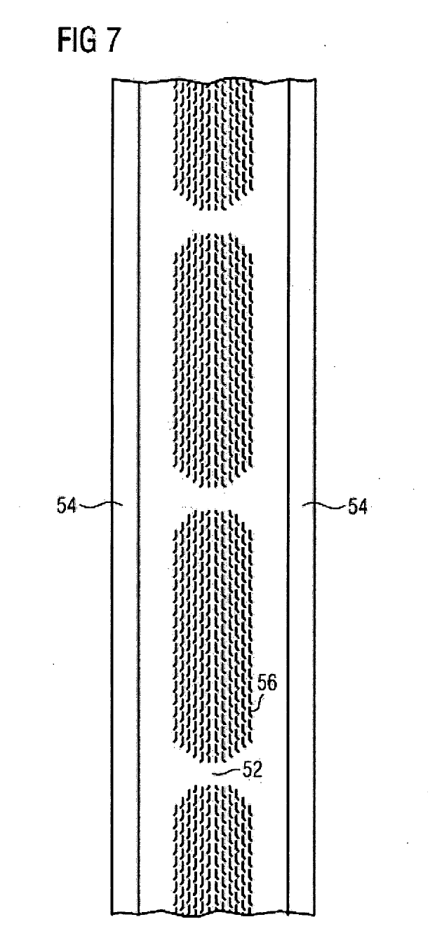

The slits 18 in the absorbent core 16 may be regularly arranged along a plurality of spaced apart lines 22 which extend in the direction parallel to the longitudinal direction, wherein these lines are spaced apart at a distance e. Two adjacent slits 18 arranged on one of these parallel lines are spaced apart by a distance c. Each slit 18 has an effective length a in longitudinal direction and an effective length d in lateral direction. The sum of the distance a and the distance c gives the length b along the longitudinal direction at which the slit-pattern along one parallel line 22 is repeated. The geometry of the slits may vary greatly, as long as the slits have an effective component extending in the longitudinal direction and an effective component extending in the lateral direction. The non-linear slits may therefore be curved, for instance in the form of waves or arcs, as is shown in FIGS. 4a-c, or may comprise two or more linear sections extending in different directions, as is shown in FIGS. 3 and 7. Also the slits may be linear slits which are at an acute angle relative to the longitudinal direction of the absorbent article, for example as shown in FIG. 4d. The pattern of slits may either extend essentially over the complete surface of the absorbent core or only selected sections thereof, for instance the central area of the absorbent core or peripheral sections such as front and back sections and/or side sections.

In FIG. 3 a selected, non-limiting example of a slit-geometry is shown from which the above outlined definitions of distances, lengths and geometry become clearer. In this pattern, each slit comprises a first linear section 18a extending in the longitudinal direction and a second linear section 18b extending in a direction at an angle α to the longitudinal direction, wherein the angle α has a value in the range between 20° and 70°, and specifically between 35° and 55°.

Generally, the effective length a of a slit in longitudinal direction can be selected to lie in the range between about 5 mm and about 20 mm. The effective length in cross direction d can be selected to lie in the range between about 0.5 mm and about 3 mm, specifically between about 1 mm and 2.5 mm. The ratio of the effective length a and the effective length d can be selected to lie in the range between 15:1 and 2:1, and, more specifically, in the range between 6:1 and 3:1. The distance e between each of the spaced apart parallel lines 22 can be selected to lie in the range between 4 and 8 mm, more specifically between 5 and 6 mm. The distance c between two adjacent slits in longitudinal direction can be selected to have a value in the range between 1 and 10 mm, specifically between 1 and 5 mm, more specifically between 1 and 3 mm.

The pattern formed by the slits 18 may define a first and a second patterned area. The arrangement of the slits 18 in the second pattern area may be a mirror inverted image of the arrangement of the slits in the first pattern area. Such a pattern may for instance arise if one imagines a mirror plane extending along the middle of the absorbent core in longitudinal direction and perpendicular to the surface of the flat absorbent core and the pattern formed by the slits on one half of the absorbent core being mirrored onto the second half of the absorbent core. In a similar manner, the slits may be mirrored along the lateral center line in the lateral direction separately or in addition to the mirroring in the longitudinal direction.

If each slit in the pattern comprises a first linear section 18a extending in longitudinal direction and a second linear section 18b extending at an angle α to the longitudinal direction, the slits within the pattern may be arranged in such a manner that the second linear sections of the respective slits are arranged along a plurality of parallel lines 24 extending in a direction at an angle α to the longitudinal direction. Such an arrangement is for instance shown in FIG. 3.

If the slits are linear slits which are at an acute angle relative to the longitudinal direction of the absorbent article, the acute is generally an angle ω, shown in FIG. 4d between about 20° and 70°. More specifically, the acute angle is about 45°. In another feature, if the linear slits have more than one direction it is desired that there is a plurality of first linear slits having a first orientation and a plurality of second linear slits having a second orientation, such that the first orientation and the second orientation are orthogonal the one another. By having the slit orthogonal to one another, an extensibility in a direction diagonal to the lateral direction and longitudinal direction may be obtained. Generally, when there is a first and second linear slits it is desirable that the each first linear slit is separated by a second linear slit, in the lateral direction, the longitudinal direction or both the longitudinal and lateral direction.

The absorbent article according to the present invention may further comprise a garment attachment system, which in one embodiment may consist of at least one adhesive strip on the surface of the liquid-impermeable backing layer 30 facing away from the wearer's body, and covering means for covering the at least one adhesive strip. The adhesive may be, for instance, a hot-melt adhesive or pressure sensitive adhesive. The covering means may be, for instance, a release tape, which may be made of a silicone paper. In a further embodiment of the absorbent article according to the present invention, the garment attachment system consists of two separate adhesive strips extending in the longitudinal direction of the absorbent article and either two corresponding release tapes or a single release tape comprising a dividing line formed by perforations, which allows the release tape to be easily divided into two separate release tapes, for covering the adhesive strips. The absorbent article comprising such a garment attachment system is also extensible when the perforated release tape or the two release tapes are still attached to the absorbent article. In the case of the perforated release tape the application of tension will divide the release tape into two separate release tapes. This allows the user to perceive the extensibility of the absorbent article even without having to remove the release tape from the absorbent article.

As shown in FIG. 2, the absorbent article 10 according to the present invention may comprise additional layers positioned between the absorbent core and the backing layer 30. The absorbent article 10 may, for instance, further comprise a transfer layer 26 for further enhancing the distribution of liquid in the horizontal direction of the absorbent article. The transfer layer 26 may, for instance, contain a meltblown material made of polypropylene fibers and having a basis weight of between 50 to 150 g/m², more specifically 70 to 110 g/m², and even more specifically 90 g/m².

Additionally or instead of the transfer layer 22, the absorbent article 10 may further comprise a layer 28 made of cellulose fluff pulp, which may be in the shape of an oval pillow, to further increase the absorbent capacity of the absorbent article and at the same time providing wearing comfort for the user.

Several specific, non-limiting embodiments of the absorbent articles according to the present invention will now be described in the following. The first of these embodiments corresponds essentially to the sanitary napkin shown in FIG. 1. In this embodiment, the liquid-permeable top layer 12 is a carded web of polypropylene fibers and has a basis weight of 27 g/m². The top layer 12 comprises oval shaped perforations 14 in the target area of the sanitary napkin. Furthermore, unlike shown in FIG. 2, the top layer may also optionally comprise embossing lines 11 in the front and back section of the sanitary napkins for further adapting its form to the shape of the wearer's body and to improve its conformability to the body of the wearer. The backing layer 30 is a layer which may be prepared from a mixture of polyethylenes having different densities.

The absorbent core 16 consists of a laminate of a nonwoven spunbond fiber layer with a basis weight of 17 g/m² made of polypropylene fibers and an airlaid fiber layer with a basis weight of 156 g/m² comprising at least 70 wt.-% cellulose fibers, from about 2 to 6 wt-% polyethylene powder, and 5 to 25 wt-% bicomponent fibers, and optionally up to 2 wt-% of a latex binder, wherein the sum of the weight percentages of the different components present in the material adds up to 100 wt-%.

In an even more specific embodiment, the combined laminate of spunbond nonwoven and airlaid fiber layer consists of 10 wt-% polypropylene, 68 wt-% cellulose fibers, 14 wt-% bicomponent fibers, 3 wt-% latex binder, and 5 wt-% polyethylene powder.

Alternatively, the nonwoven spunbond layer is laminated to a coform layer containing 40 wt-% polypropylene and 60 wt-% cellulose. The absorbent core 16 comprises a pattern of slits 18 as outlined in the following. This pattern may either cover all of the surface of the absorbent core or only the central area or only peripheral areas of the absorbent core 16.

On the outside surface of the backing layer 30, the absorbent article further comprises a garment attachment system consisting either of two separate adhesive strips running in longitudinal direction, each covered with a release tape 32 made of silicon paper. Alternatively, a single release tape 32 made, for instance, of a silicone paper and comprising an easily dividable line of perforations may be used.

In a further specific embodiment the above described sanitary napkins further comprise a transfer layer 26 and a fluff layer 28 positioned between the absorbent core 16 and the backing layer 30. The transfer 26 layer is a rectangular meltblown layer of polypropylene with a basis weight of 90 g/m² and has a length of 12.5 mm and a width of 50 mm. Positioned underneath the transfer layer 26 is the pad 28 of cellulose fluff. In a further embodiment the sanitary napkin only additionally comprises the transfer layer 26, but not the fluff pad 28.

As outlined above, the present invention provides in a second aspect an absorbent core for use in an absorbent article having a longitudinal direction and a lateral direction, wherein the absorbent core is made of a nonwoven absorbent material and comprises a pattern of slits formed therethrough. Therein each slit is formed by removing a section of the nonwoven absorbent material from the absorbent core and simultaneously densifying the nonwoven absorbent material in the areas of the absorbent core adjacent to the edge of the opening created by slitting the core material, i.e. adjacent to the removed section of the slit.

The absorbent core may be any suitable material for use in absorbent articles such as sanitary napkins. The special slits according to the second aspect of the present invention can especially be formed in the above described absorbent cores discussed in connection with the absorbent articles according to the first aspect of the present invention. It should therefore be understood that the absorbent articles described above as the first aspect of the present invention may have the specially slitted absorbent cores according to the second aspect of the present invention and that the absorbent cores described with references to the second aspect of the present invention may be made of the materials described with reference to the absorbent cores in the absorbent articles according to the first aspect of the present application. The material for the absorbent cores according to the second aspect of the present invention may therefore be selected from the group consisting of absorbent materials comprising a layer of material having a basis weight of between 20 g/m² to 1000 g/m², which is selected from the group consisting of airlaid fiber layers, coform fiber layers, meltblown fiber layers or laminates thereof and laminates of airlaid fiber layers, coform fiber layers or meltblown fiber layers or laminates thereof with a nonwoven support. The ratio of tensile strength in longitudinal direction and tensile strength in lateral direction of the absorbent cores according to the second aspect of the present invention may be selected to be in the range from 3.5:1 to 6:1.

Furthermore, it should be understood that the slits and the slit patterns according to the second aspect of the present invention may have the same geometry or arrangement as those described with reference to the absorbent articles according to the first aspect of the present invention.

By providing the special slits 18 in the absorbent core 16, the extensibility and flexibility of the absorbent core 16 is increased. This is achieved not only by dividing the absorbent material in the area of the slits 18, but additionally by removing absorbent material from within the area of the slit, for instance as a thin strip of material corresponding to the respective form of the strip. This may provide for a quicker intake of liquid through the absorbent core. Additionally, the nonwoven material in the areas of the absorbent core adjacent to the removed sections of the slit are densified, thereby defining areas in which the capillarity of the nonwoven material is increased. This results in an improved liquid transport along the slits formed in the nonwoven absorbent material without having to provide additional embossing lines, and leads to an improved longitudinal liquid distribution in the absorbent core 16. The special slits therefore enable a more efficient use of the absorbent capacity of the nonwoven material used in the absorbent core 16.

In a further embodiment of the absorbent core according to the present invention, the formation of the slits in the absorbent core increases the distribution of defibrinated sheep blood in the longitudinal direction of the absorbent core by 1.5 to 2 times, compared to an identical absorbent core, but which comprises a pattern of cuts corresponding to the pattern of slits, as measured by observing the distribution of 3 ml of defibrinated sheep blood, which have been brought into contact with the absorbent core, after 10 minutes. “Cuts” in this respect are understood as meaning cuts which have been formed in the absorbent material using a conventional, sharp cutting element, i.e. blade.

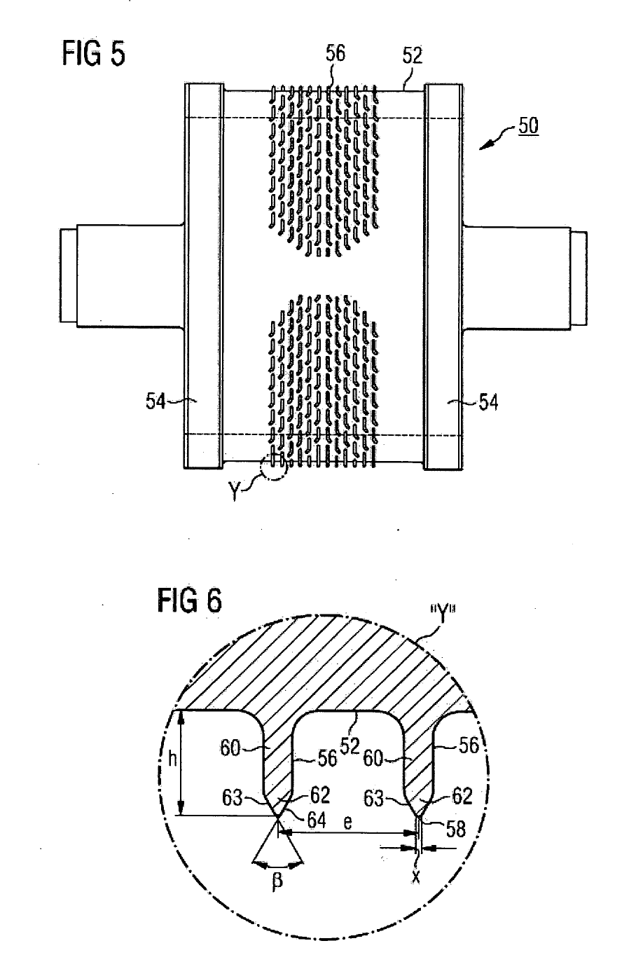

To form the special slits in the absorbent core for use in the absorbent article, the present invention provides a special punching die. A non-limiting embodiment of such a punching die is shown in FIGS. 5 to 7. The punching die shown in FIG. 5 is in the form of a roller 50 having an essentially cylindrical shape and having an outer surface 52. The punching die may further comprise, as shown in FIG. 5, two outer, elevated rim sections 54. The punching die comprises at least one plurality of punching elements 56 for forming a pattern of slits 20 in an absorbent core. The height h of the punching elements 56, which is the distance between the outer surface 52 and the tips 58 of the punching elements 56, can be adapted for each respective absorbent material to be cut. Each punching element comprises a base section 60 protruding from the outer surface 52 of the roller 50. In the embodiment shown in FIG. 5 and FIG. 6, the latter one showing a cross section of the area denoted “Y” in FIG. 5, the side walls 60 of the punching element 56 are essentially linear and extend in a direction perpendicular to the outer surface 52 of the roller 50. Extending from the base section 60 is the blade section 62 of the punching element, which has a trapezoidal cross section perpendicular to the direction in which the respective slit in the pattern is formed, and a tip 58. The trapezoidal cross section defines two parallel sides running in parallel with the outer surface 52 and two non-parallel sides, said two non-parallel sides enclosing an angle β of between 45 to 75°, preferably between 55° and 65°, and the smaller of said two parallel sides, which represents the tip 58 of the punching element, has a length x of between 0.2 mm and 0.45 mm.

In an embodiment of the punching die according to the present invention, the distance x is selected to be 0.25 mm and the angle β is selected to be 60°.

Each punching element provides two parallel cutting edges formed at the edges defined by the tip 58 and the two non-parallel sides 62 and 63 of the blade section. The angle β between the two non-parallel sides is sufficiently small for the two edges to act as cutting edges so that thin slits of material are removed from the absorbent material. At the same time, angle β is sufficiently large so that the punching element can densify the absorbent material adjacent to the punched or cut out slits.

FIG. 7 shows a projection of the outer surface 52 onto a plane. From this figure it becomes apparent that the punching die shown in FIG. 5 comprises three pluralities of punching elements 56 forming three identical patterns 20 on the outer surface 52 of the roller 50.

In the process according to the present invention, a web of a nonwoven material for forming the absorbent core is provided and a pattern of slits is formed in the web using the above described punching die. Accordingly, the present invention also provides absorbent cores and articles obtainable by the process according to the present invention.

In the process according to the present invention all the materials described above with reference to the first and second aspects of the present invention can be used for forming the respective components of the absorbent article. The process according to the present invention utilizes the punching die according to the present invention for forming a pattern of slits in the absorbent core of the absorbent article.

Accordingly, the present invention provides a process for forming an absorbent article, the process comprising the steps of: a) providing an absorbent core material sheet for forming an absorbent core, b) forming a pattern of slits in the absorbent core material sheet with a punching die comprising a plurality of punching elements for forming the pattern of slits, each punching element comprising a blade section having a trapezoidal cross section perpendicular to the direction in which the slit is formed, wherein the trapezoidal cross section defines two parallel sides and two non-parallel sides, said two non-parallel sides enclosing an angle β of between 45 to 75°, preferably between 55° and 65°, and the smaller one of said two parallel sides represents the tip of the punching element and has a length of between 0.2 mm and 0.45 mm, and c) cutting an absorbent core from absorbent core material such that the pattern of slits is located in the absorbent core.

In a specific embodiment of the process according to the present invention, the angle β enclosed by the two non-parallel sides of the trapezoid is 60° and the smaller of the two parallel sides has a length of 0.25 mm.

In further embodiments of the process according to the present invention, the process may comprise the additional steps of d) attaching the absorbent core to a top layer material sheet, e) laminating a backing layer material sheet to the top layer material sheet in such a manner that the absorbent core is positioned between the top layer material layer and the backing material layer, and f) cutting the laminate of top layer material sheet, absorbent core and backing layer material sheet into the shape of an absorbent article.

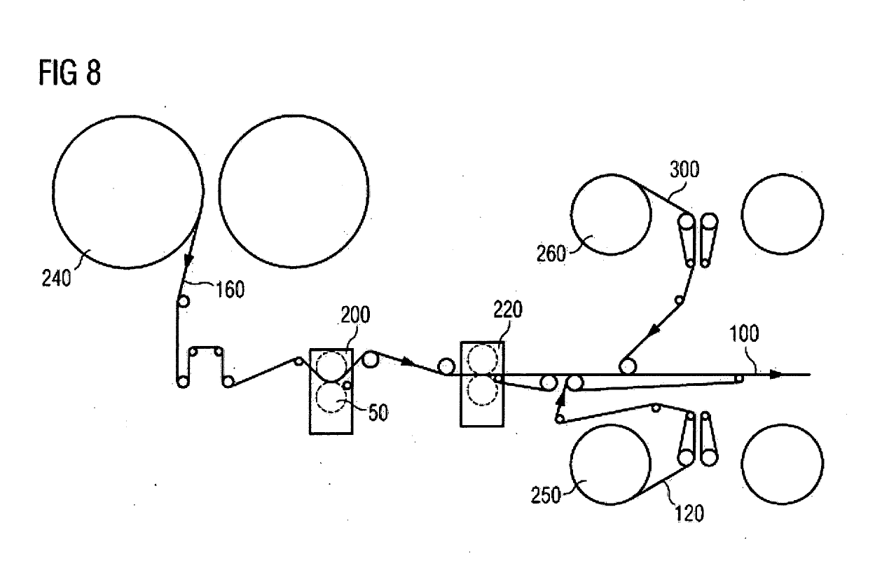

FIG. 8 shows a schematic representation of an embodiment of the process according to the present invention. Except for the use of the punching die according to the present invention, the process may use known apparatuses and process steps commonly used in the art of producing absorbent articles, which are well known to the person skilled in the art and therefore do not require a detailed discussion.

The absorbent core material sheet 160 is provided from supply roll 240 and transported to slitting station 200. The slitting station 200 comprises the punching die 50 according to the present invention. In the slitting station, patterns of slits are formed in the absorbent core material sheet 160. From the slitting station 200, the slitted absorbent core material sheet 160 is transported to the cutting station 220, which cuts absorbent cores from the absorbent core material sheet 160. These absorbent cores are then transported from the cutting station 220 and attached, for instance laminated, to a top layer material sheet 120, which is provided from supply roll 250. The remaining absorbent core material sheet can be removed separately from the cutting station using conventional techniques (not shown). To laminate the absorbent core to the top layer material sheet 120, an adhesive may be applied to the top layer material sheet 120 before the absorbent cores are deposited on the top layer material sheet 120. Next, a backing layer material sheet 300, which is provided from supply roll 260 is laminated to the laminate of absorbent cores and top layer material sheet 120 in such a manner that the absorbent cores are positioned between top layer material sheet 120 and the backing layer material sheet 300. Again, an adhesive may be applied to the backing layer material sheet 300 to achieve the lamination.

From this laminate, absorbent articles comprising a top layer 12, absorbent core 16 and backing layer 30 can subsequently be cut and formed. If additional layers, such as transfer layers and fluff pads are to be incorporated into the absorbent articles, additional conventional process steps would have to be incorporated into the process, by means of which these components are formed in the absorbent articles. The process may further comprise embossing steps to provide embossing lines or patterns in the absorbent articles.

Furthermore, steps for forming garment attachment systems on the absorbent article, either on the backing sheet or the wing sections of the absorbent articles or both may be carried out, for instance application of adhesive strips and application of corresponding release tapes.

### EXAMPLES

1. Liquid Transport in Slit Absorbent Cores

To test the improved liquid transportation properties of the absorbent cores and their mechanical properties, a laminated web consisting of a spunbonded support layer having a basis weight of 17 g/m² and an airlaid layer having a basis weight of 156 g/m², resulting in a combined basis weight of the absorbent material of 173 g/m².

These nonwoven webs were then slitted using a punching die according to the present invention, wherein the pattern of the punching dies was those shown in FIGS. 5 and 7. The length x at the tip of the punching dies was 0.25 mm and the angle β enclosed by the two non-parallel sides of the blade section was 60°. The pattern formed by the punching dies within one plurality of punching dies is characterized by the following dimensions shown below in Table 1:

TABLE 1
Slit pattern parameters

| | [mm] |
|---|---|
| Parameter | |
| a (effective length in MD) | 8.7 |
| b (repeat length in MD) | 10.0 |
| c (distance between two adjacent slits in MD) | 1.3 |
| d (effective length in CD) | 2.2 |
| e (distance between adjacent pattern lines in MD) | 5 |
| | [°] |
| α (angle between first and second linear section) | 40 |

Slit absorbent core materials obtained by this method are in the following denoted as Core Type 1.

In a comparative example, an identical nonwoven web was cut using conventional, sharp cutting knifes arranged in the same pattern. The thus obtained cut absorbent core materials are in the following denoted as Core Type 2 (Control).

The different absorbent cores were then placed on a liquid-impermeable sheet and 3 ml of defibrinated sheep blood obtained from OXOID, Wesel, Germany, were applied onto the central area of each absorbent core using a pipette. After 10 minutes, the maximum lateral and longitudinal wicking distance of the blood in the absorbent cores were measured. In Table 2 below the observed wicking distances, derived as the average values from measuring 10 samples, are listed:

TABLE 2
Measured wicking distances in longitudinal and lateral direction

| | | Core Type 2 |
|---|---|---|
| | Core Type 1 | (Control) |
| max. longitudinal length γ [mm] | 90 | 54 |
| max. lateral length δ [mm] | 50 | 48 |
| γ/δ | 1.8 | 1.1 |

The above data shows that the absorbent cores obtained using the punching die according to the present invention exhibit an improved liquid transportation in the longitudinal direction of the absorbent core. The absorbent cores therefore make more efficient use of the absorbent capacity of the absorbent material. At the same time, the liquid transportation in lateral direction is only increased to a small extend. A too high transport rate in the lateral direction would increase the risk of leakage at the sides of the absorbent article. The improved liquid transportation in longitudinal direction therefore enables it that more of the absorbent material present in the absorbent article is utilized before the absorbent article needs to be changed.

2. Mechanical Properties of the Absorbent Cores According to the Present Invention.

The tensile strength and elongation at tear of the samples according to the present invention and the Control-samples were measured using a conventional tensile strength machine complying to DIN 51221. The samples had a width of 50 mm and were measured with a clearance between clamps of 50 mm and a haul-off speed of 50 mm/min. The measurement followed essentially the procedure outlined in DIN 53857 but did not use a pre-load force. The flexural resistance of the absorbent core samples was measured according to the above mentioned circular bend procedure.

The measured values of the samples according to the present invention are compared below in Table 3 with those of an identical absorbent core without any slits formed therein [(Core Type 3 (Control)]. All values are average values derived from measurement of 10 samples.

TABLE 3
Mechanical properties of the absorbent cores

| | | Core Type 3 |
|---|---|---|
| Test specimen | Core Type 1 | (Control) |
| Tensile strength MD (N/50 mm) | 23.2 | 60.0 |
| Tensile strength CD (N/50 mm) | 5.1 | 44.7 |
| Elongation at tear MD (%) | 12.5 | 19.3 |
| Elongation at tear CD (%) | 37 | 19 |
| Flexural resistance (N) | 0.80 | 1.79 |

The above data shows that the absorbent cores comprising the slits have an increased extensibility whilst maintaining a sufficient flexural resistance and tensile strength in MD for processing the material in a process for making absorbent articles.

While the present invention has in the above been described with special reference to specific embodiments and examples, those skilled in the art will readily appreciate that many modifications are possible in the exemplary embodiments without materially departing from the novel teachings and advantages of this invention. Accordingly, all such modifications are intended to be included within the scope of this invention as defined in the following claims.

It should be further noted that any patents, applications or publications referred to herein are incorporated by reference in their entirety.

What is claimed:

1. An absorbent article, having a longitudinal direction and a lateral direction, said absorbent article comprising:
a) a liquid-permeable top layer, wherein the top layer has an extensibility in lateral direction,
b) a liquid-impermeable backing layer laminated to the top layer, and
c) an absorbent core positioned between the top layer and the backing layer, wherein said absorbent core comprises an absorbent nonwoven web comprising a plurality slits therethrough, wherein the plurality of slits are selected from the group consisting of non-linear slits, linear slits which are positioned at an acute angle relative to the longitudinal direction of the absorbent article and mixtures thereof.

2. The absorbent article according to claim 1, wherein the absorbent core material has a basis weight of between 20 g/m² and 1000 g/m².

3. The absorbent article according to claim 2, wherein the absorbent core material has a basis weight of between 100 g/m² and 200 g/m².

4. The absorbent article according to claim 2, wherein the absorbent nonwoven web is selected from the group consisting of airlaid fiber layers, coform fiber layers, meltblown fiber layers or laminates thereof and laminates of airlaid fiber layers, coform fiber layers or meltblown fiber layers or laminates thereof with a nonwoven support.

5. The absorbent article according to claim 4, wherein the nonwoven support in the absorbent core is a spunbond fiber layer with a basis weight in the range of 10 to 40 g/m².

6. The absorbent article according to claim 5, wherein the absorbent core material comprises a laminate of an airlaid fiber layer or a coform fiber layer with a basis weight of 140 g/m² to 170 g/m² and a spunbond fiber layer with a basis weight in the range of 10 to 25 g/m².

7. The absorbent core according to claim 1, wherein each non-linear slit has an effective length in longitudinal direction (a) in the range between about 5 mm and about 20 mm and an effective length in lateral direction (d) in the range between about 0.5 mm and about 3 mm.

8. The absorbent core according to claim 7, wherein the ratio of the effective length in longitudinal direction (a) and the effective length in lateral direction (d) is in the range between 15:1 and 2:1.

9. The absorbent article according to claim 7, wherein the non-linear slits are arranged essentially regularly along a plurality of spaced apart lines in parallel with the longitudinal direction, wherein the distance between each of the respective spaced apart parallel lines is in the range between 4 and 8 mm.

10. The absorbent article according to claim 9, wherein the distance between two adjacent slits in longitudinal direction (c) is in the range between 1 mm and 10 mm.

11. The absorbent article according to claim 10, wherein the distance between two adjacent slits in the longitudinal direction (c) is in the range between 1 and 5 mm.

12. The absorbent article according to claim 1, wherein each non-linear slit comprises a first linear section extending in longitudinal direction and a second linear section extending in a direction at an angle α to the longitudinal direction, wherein the angle α is in the range between 20° and 70°.

13. The absorbent article according to claim 12, wherein the angle α is in the range between α is in the range between 35° and 55°.

14. The absorbent article according to claim 13, wherein the non-linear slits defines a first and a second pattern area, and wherein the arrangement of the slits in the second pattern area is a mirror inverted image of the arrangement of the slits in the first pattern area.

15. The absorbent article according to claim 14, wherein the non-linear slits in each of the respective pattern sections are arranged in such a way that the second linear sections of the respective slits are arranged along a plurality of parallel lines extending in a direction at the angle α to the longitudinal direction.

16. The absorbent article according to claim 1, wherein the absorbent article comprises a sanitary napkin.

17. The absorbent article according to claim 16, wherein the article further comprises a garment attachment system, preferably consisting of at least one adhesive strip on the surface of the liquid-impermeable backing layer facing away from the wearer's body and covering means for covering the at least one adhesive strip.

18. The absorbent article according to claim 17, wherein the garment attachment system consists of two separate adhesive strips extending in the longitudinal direction of the sanitary napkin and either two corresponding release tapes or a single release tape comprising a line of perforations dividing the single release tape into two sections corresponding to the adhesive strips.

19. The absorbent article according to claim 16, further comprising side wings, each side wing comprising a wing attachment system for attaching the wings to the garment.

20. The absorbent article according to claim 1, wherein the non-linear slits in the absorbent core are made by using a punching die comprising a plurality of punching elements for forming the pattern of slits, each punching element comprising a blade section having a trapezoidal cross section perpendicular to the direction in which the slit is formed, wherein the trapezoidal cross section defines two parallel sides and two non-parallel sides, said two non-parallel sides enclosing an angle β of between 45 to 75°, and the smaller of said two parallel sides represents the tip of the punching element and has a length of between 0.2 mm and 0.45 mm.

21. The absorbent article according to claim 1, further comprising a transfer layer and, optionally, a fluff pad positioned between the absorbent core and the backing sheet.

22. An absorbent core for use in an absorbent article having a longitudinal direction and a lateral direction, wherein the absorbent core is made of a nonwoven material and comprises a pattern of slits formed therethrough, wherein each slit is formed by removing a section of the nonwoven material from the absorbent core and simultaneously increasing the density of the nonwoven material of the absorbent core in the areas adjacent to the removed section of absorbent material.

23. The absorbent core according to claim 22, wherein the formation of the slits in the absorbent core increases the distribution of defibrinated sheep blood in the longitudinal direction of the absorbent core by 1.5 to 2 times, compared to an identical absorbent core comprising a pattern of cuts corresponding to the pattern of slits instead of the slits, as measured by observing the distribution of 3 ml of defibrinated sheep blood after 10 minutes.

24. The absorbent core according to claim 22, wherein the absorbent core comprises a layer of material having a basis weight of between 20 g/m² to 1000 g/m² and is selected from the group consisting of airlaid fiber layers, coform fiber layers, meltblown fiber layers or laminates thereof and laminates of airlaid fiber layers, coform fiber layers or meltblown fiber layers or laminates thereof with a nonwoven support.

25. The absorbent core according to claim 22, wherein the absorbent core comprises a plurality of non-linear slits therethrough, such that the ratio of the tensile strength of the absorbent core in longitudinal direction and the tensile strength of the absorbent core in lateral direction is in the range from 3.5:1 to 6:1.

26. The absorbent article according to claim 22, wherein the absorbent core material has a basis weight of between 100 g/m² and 200 g/m².

27. The absorbent article according to claim 24, wherein the nonwoven support of the absorbent core material is a spunbond fiber layer with a basis weight in the range of 10 to 40 g/m².

28. The absorbent article according to claim 24, wherein the absorbent core material is a laminate of an airlaid fiber layer or a coform fiber layer with a basis weight of 140 g/m² to 170 g/m and a spunbond fiber layer with a basis weight in the range of 10 to 25 g/m².

29. The absorbent core according to claim 25, wherein each non-linear slit has an effective length in longitudinal direction (a) in the range between about 5 mm and about 20 mm and an effective length in lateral direction (d) in the range between about 0.5 mm and about 3 mm.

30. The absorbent core according to claim 29, wherein the ratio of the effective length in longitudinal direction (a) and the effective length in lateral direction (d) is in the range between 15:1 and 2:1.

31. The absorbent core according to claim 30, wherein the non-linear slits are arranged essentially regularly along a plurality of spaced apart lines in parallel with the longitudinal direction, wherein the distance between each of the respective spaced apart parallel lines is in the range between 4 and 8 mm.

32. The absorbent core according to claim 31, wherein the distance between two adjacent slits in longitudinal direction (c) is in the range between 1 mm and 10 mm.

33. The absorbent core according to claim 22, wherein each slit comprises a first linear section extending in longitudinal direction and a second linear section extending in a direction at an angle α to the longitudinal direction, wherein the angle α is in the range between 20° and 70°.

34. The absorbent core according to claim 33, wherein the pattern formed by the slits defines a first and a second patter area, and wherein the arrangement of the slits in the second pattern area is a mirror inverted image of the arrangement of the slits in the first pattern area.

35. The absorbent core according to claim 34, wherein the slits in each of the respective pattern sections are arranged in such a way that the second linear sections of the respective slits are arranged along a plurality of parallel lines extending in a direction at an angle α to the longitudinal direction.

36. The absorbent core according claim 22, wherein the slits in the absorbent core are made by using a punching die comprising a plurality of punching elements for forming the pattern of slits, each punching element comprising a blade section having a trapezoidal cross section perpendicular to the direction in which the slit is formed, wherein the trapezoidal cross section defines two parallel sides and two non-parallel sides, said two non-parallel sides enclosing an angle β of between 45 to 75°, and the smaller of said two parallel sides represents the tip of the punching element and has a length of between 0.2 mm and 0.45 mm.

37. A punching die for forming slits into an absorbent material for use in an absorbent article, comprising at least one plurality of punching elements for forming a pattern of slits in said absorbent core, each punching element comprising a blade section having a trapezoidal cross section perpendicular to the direction in which the respective slit in the pattern is formed, wherein the trapezoidal cross section defines two parallel sides and two non-parallel sides, said two non-parallel sides enclosing an angle β of between 45 to 75°, and the smaller of said two parallel sides represent the tip of the punching element, and has a length of between 0.2 mm and 0.45 mm.

38. The punching die according to claim 37, wherein the angle β enclosed by the two non-parallel sides of the trapezoid is 60° and the smaller of the two parallel sides has a length of 0.25 mm.

39. The punching die according to claim 37, wherein the punching die is in the form of an essentially cylindrical roller comprising an outer surface, and the at least on plurality of punching elements is located on the outer surface of the roller.

40. A process for forming an absorbent article, the process comprising the steps of:
a) providing an absorbent core material sheet for forming an absorbent core,
b) forming a pattern slits in the absorbent core material sheet with a punching die comprising a plurality of punching elements for forming the pattern of slits, each punching element comprising a blade section having a trapezoidal cross section perpendicular to the direction in which the slit is formed, wherein the trapezoidal cross section defines two parallel sides and two non-parallel sides, said two non-parallel sides enclosing an angle of between 45 to 75°, and the smaller of said two parallel sides represents the tip of the punching element and has a length of between 0.2 mm and 0.45 mm,
c) cutting an absorbent core from absorbent core material such that the pattern of slits is located in the absorbent core.

41. The process according to claim 40, wherein the wherein the angle enclosed by the two non-parallel sides of the trapezoid is 60° and the smaller of the two parallel sides has a length of 0.25 mm.

42. The process according to claim 40, wherein the process further comprises the steps of
d) attaching the absorbent core to a top layer material sheet,
e) laminating a backing layer material sheet to the top layer material sheet in such a manner that the absorbent core is positioned between the top layer material layer and the backing material layer, and
f) cutting the laminate of top layer material sheet, absorbent core and backing layer material sheet into the shape of an absorbent article.

43. The absorbent article according to claim 1, wherein the ratio of the tensile strength of the absorbent core in longitudinal direction and the tensile strength of the absorbent core in lateral direction is in the range from 3.5:1 to 6:1.

44. The absorbent article according to claim 43, wherein the wherein the absorbent core material has a basis weight of between 20 g/m² and 800 g/m², the absorbent nonwoven web is selected from the group consisting of airlaid fiber layers, coform fiber layers, meltblown fiber layers or laminates thereof and laminates of airlaid fiber layers, coform fiber layers or meltblown fiber layers or laminates thereof with a nonwoven support

45. The absorbent article according to claim 1, wherein the slits comprise linear slits positioned at an acute angle relative to the longitudinal direction of the absorbent articles, wherein the acute angle comprises an angle between 20° and 70°.

46. The absorbent article according to claim 45, wherein the acute angle is 45°.

47. The absorbent article according to claim 45, wherein the slits comprise a plurality of first linear slits having a first orientation and a plurality of second linear slits having a second orientation, such that the first orientation and the second orientation are orthogonal the one another.

48. The absorbent article according to claim 47, wherein each first linear slit is separated by a second linear slit, in the lateral direction.

49. The absorbent article according to claim 48, wherein each first linear slit is separated by a second linear slit, in the longitudinal direction.

50. The absorbent article according to claim 47, wherein each first linear slit is separated by a second linear slit, in the longitudinal direction.

51. The absorbent article according to claim 1, wherein the extensibility on the top layers is at least 30% to 70% at 4N/50 mm and wherein the backing layer has a higher extensibility in lateral direction than in longitudinal direction and the extensibility of the backing layer in lateral direction is at least 5% to 40% at 4N/50 mm.
